(19) United States
(12) Patent Application Publication
(10) Pub. No.: US 2025/0241981 A1
(43) Pub. Date: Jul. 31, 2025

(54) NOVEL COMPOSITION

(71) Applicant: Arecor Limited, Little Chesterford (GB)

(72) Inventors: Jan JEZEK, Little Chesterford (GB); David GERRING, Little Chesterford (GB); Sarah HOWELL, Little Chesterford (GB); Jorge PINTO, Little Chesterford (GB)

(21) Appl. No.: 19/185,396

(22) Filed: Apr. 22, 2025

Related U.S. Application Data
(63) Continuation of application No. 16/956,382, filed on Jun. 19, 2020.

(30) Foreign Application Priority Data
Dec. 22, 2017 (GB) 1721846.2

(51) Int. Cl. A61K 38/12; A61K 9/08; A61K 38/08; A61K 38/095; A61K 47/10; A61K 47/12; A61K 47/18; A61K 47/20; A61K 47/22; A61K 47/26

(52) U.S. Cl. CPC A61K 38/12; A61K 9/08; A61K 38/08; A61K 38/095; A61K 47/10; A61K 47/12; A61K 47/183; A61K 47/20; A61K 47/22; A61K 47/26

(57) ABSTRACT

There is provided, inter alia, an aqueous solution composition of pH in the range 4.0-7.5 comprising: a peptide therapeutic agent; optionally one or more buffers being substances having at least one ionisable group with a pK a in the range 3.0 to 8.5 and which pk a is within 2 pH units of the pH of the composition; and a stabilizer; wherein the peptide therapeutic agent does not contain ionisable groups with pk a in the range 3.0 to 8.5, and wherein the buffers are present in the composition at a total concentration of 0-5 mM.

This invention relates to aqueous solution compositions of therapeutic agents, in particular peptides, at low buffer concentrations.

### BACKGROUND

A large number of therapeutic agents are peptides. Despite the increasing number of protein-based pharmaceuticals amongst marketed and pipeline development products, peptides continue to play an important role due to their lower production complexity, production cost and good efficacy and tolerability in humans.

When formulated as aqueous solutions, peptides are unstable and are susceptible to structural degradation and consequent loss of biological activity while stored. The structural degradation is typically chemical in nature, including hydrolytic cleavage, cyclic imide formation, isomerization or oxidation. In some cases, the degradation can be physical in nature (e.g. aggregation or gel formation), although these processes are relatively less common in peptides than in the case of larger proteins.

The rates of the degradation processes are proportional to temperature, and peptide therapeutics are generally more stable at lower temperatures. However, to ensure convenience for patients there is often need to develop products that are stable at elevated temperatures, such as up to 25° C. or up to 30° C., either for a specific period of time or for the entire shelf-life.

One of the most critical parameters to control the stability of peptide therapeutics is pH. Therefore, pH optimization is a key step in formulation development. M any therapeutic peptides are formulated at a selected pH between 4.0-7.5. It is thought to be important to ensure that the pH is maintained at the selected value and pH fluctuations are minimized. Therefore, it has been understood that a certain degree of buffering capacity is needed in the formulation. Whilst some peptide therapeutic agents provide buffering capacity themselves in the pH range between 4.0-7.5 (i.e. are self-buffering), others do not. Generally, peptides that comprise at least one ionisable group with pk a between about 3.0-8.5 provide buffering capacity at least in part of the pH range between 4.0-7.5. In such cases the buffering capacity is substantially provided within around 1 pH unit of the pK a of the ionisable group. It is true that some buffering capacity is provided by use of a molecule having at least one ionisable group having a pK a more than 1 pH unit away from the pH of the composition (e.g. within around 2 pH units of the pH), however, a significantly higher concentration of buffer molecule would typically be required to have a comparable pH stabilising effect as compared with use of a buffer molecule having an ionisable group with a pk a closely matched to the pH of the composition.

In the case of peptides composed of natural amino acids, some buffering capacity in at least part of the pH range between 4.0-7.5 can be expected if side chains of free aspartic acid, free glutamic acid (i.e. aspartic acid and glutamic acid side chains wherein the side chain is not engaged in an amide or other bond), histidine or free cysteine (i.e. cysteine side chains that are not engaged in a disulphide or other bond) are part of the primary amino acid sequence. In such cases, buffering capacity is substantially provided in a pH range within around 1 unit of the pK a of the above amino acid(s) present in the peptide structure. Peptides that do not comprise any of these four amino acid side chains do not provide any meaningful buffering capacity in the pH range 4.0-7.5 unless they comprise an ionisable non-peptide moiety that can contribute to the buffering capacity. Ionisable groups that may be present at the N-terminus and/or C-terminus of a peptide typically have pk a values of less than 3.0 (carboxyl group at the C-terminus) and greater than 8.5 (amino group at the N-terminus) and therefore do not provide substantial buffering capacity in the pH range 4.0-7.5.

The present invention addresses the problem of stability of peptide therapeutic agents in aqueous solution compositions, in particular those that do not provide any substantial buffering capacity at pH range 4.0-7.5.

Liquid formulations of peptide therapeutic agents may be intended for different routes of administration, including intravenous (IV), subcutaneous (SC), intramuscular (IM) and oral administration. Depending on the particular administration routes certain formulation parameters are required or preferred. For example, it is strongly preferable if formulations intended for SC and IM administration are approximately isotonic (e.g. 200-500 mOsm/l, preferably around 300 mOsm/l). In contrast, IV products, particularly those that are delivered following dilution in an IV bag can be strongly hypotonic or hypertonic if required.

WO2006/138181A 2 (AM GEN, INC.) discloses self-buffering protein formulations which are substantially free of other buffering agents.

US2016/0106840A 1 (EAGLE PHARMACEUTICALS INC.) discloses bivalirudin compositions which have a pH of about 3 to about 5 and are substantially free of buffer.

U.S. Pat. No. 8,420,081B2 (ABBVIE INC.) discloses aqueous formulations of antibodies wherein the formulations have conductivity of less than about 2.5 mS/cm.

WO2008/084237 (ARECOR LIMITED) discloses protein compositions which do not comprise conventional buffers in a meaningful amount. Instead “displaced buffers” which are additives with pkₐ values at least 1 unit less than or 1 unit greater than the pH of the composition are utilised.

### SUMMARY OF THE INVENTION

Thus, according to the present invention, there is provided an aqueous solution composition of pH in the range 4.0 to 7.5 comprising:
a peptide therapeutic agent;
optionally one or more buffers being substances having at least one ionisable group with a pkₐ in the range 3.0 to 8.5 and which pkₐ is within 2 pH units of the pH of the composition; and
a stabilizer;
   wherein the peptide therapeutic agent does not contain ionisable groups with pkₐ in the range 3.0 to 8.5, and wherein the buffers are present in the composition at a total concentration of 0-5 mM.

### FIGURES

FIG. 1: Structure of vasopressin.

FIG. 2: Structure of desmopressin.

FIG. 3: Structure of lypressin.

FIG. 4: Structure of atosiban.

FIG. 5: Structure of carbetocin.

FIG. 6: Structure of oxytocin.

FIG. 7: Structure of octreotide.

FIG. 8: Structure of caspofungin.

FIG. 9: Structure of anidulafungin.

FIG. 10: Structure of micafungin.

FIG. 11: Structure of terlipressin.

### DETAILED DESCRIPTION OF THE INVENTION

Described herein are stable aqueous solution compositions of peptide therapeutic agents having no or very low concentration of buffer.

It should be noted that all references herein to “pH” refer to the pH of a composition evaluated at 25° C. All references to “pKa” refer to the pKa of an ionisable group evaluated at 25° C. (see CRC Handbook of Chemistry and Physics, 79th Edition, 1998, D. R. Lide). If required, pk a values of amino acid side chains as they exist in a peptide can be estimated using a suitable calculator.

The present inventors have discovered that buffers have a detrimental impact on the stability of peptide therapeutic agents, in a concentration-dependent manner. Therefore, the concentration of buffer in the composition should be limited as much as possible.

The buffer(s) where present will have buffering capacity at the pH of the composition. Buffers typically comprise ionisable groups with pk a within 1 pH unit of the pH of the composition, however, a moiety which has ionisable groups with pk a 1 pH unit greater or less than the pH of the composition may also provide some buffering effect if present in a sufficient amount. In one embodiment, the (or a) buffer comprises ionisable groups with pk a within 1 pH unit of the pH of the composition. In another embodiment, the (or a) buffer comprises ionisable groups with pk a within 1.5 pH units of the pH of the composition (such as between 1 and 1.5 pH units of the pH of the composition). In a further embodiment, the (or a) buffer comprises ionisable groups with pk a within 2 pH units of the pH of the composition (such as between 1.5 and 2 pH units of the pH of the composition).

In an embodiment, the composition does not contain a buffer. In an embodiment, the composition contains a single buffer. In an embodiment, the composition contains two buffers.

In one embodiment, the total concentration of buffers in the composition is less than 5 mM, such as less than 4 mM, less than 3 mM, less than 2 mM, less than 1 mM, less than 0.5 mM, less than 0.4 mM, less than 0.3 mM or less than 0.2 mM. In one embodiment, the total concentration of buffers is 0.1-5 mM, such as 0.5-5 mM, 0.1-4 mM, 0.5-4 mM, 0.1-3 mM, 0.5-3 mM, 0.1-2 mM, 0.5-2 mM, 0.1-1 mM or 0.5-1 mM. In one embodiment, the aqueous solution composition is substantially free of buffer. As used herein, “substantially free” means the aqueous solution composition contains less than 0.1 mM of buffer. When considering the concentration of buffer in solution, any buffering capacity of the peptide therapeutic agent itself should be excluded.

The pH of an aqueous solution decreases if an acid is added and increases if a base is added. At a given temperature and atmospheric pressure, the magnitude of the pH decrease on addition of an acid or the magnitude of the pH increase on addition of a base depends on (1) the amount of the acid or the base added, (2) the starting pH of the aqueous solution (i.e. prior to the addition of the acid or the base) and (3) the presence of a buffer. Thus, (1) starting from a given pH, the addition of a greater amount of an acid or a base will result in greater magnitude of pH change, (2) addition of a given amount of an acid or a base will result in the greatest pH change at neutral pH (i.e. pH 7.0) and the magnitude of the pH change will decrease as the starting pH moves away from pH 7.0 and (3) the magnitude of the pH change, starting from a given pH, will be smaller in the presence of a buffer than in the absence of a buffer. A buffer thus has the ability to reduce the change in pH if an acid or a base is added to the solution.

Suitably, a substance is considered to be a buffer if it is capable of reducing the magnitude of the pH change of a solution to 75%, preferably 50%, most preferably to 25%, compared with an identical solution that does not comprise the buffer, when either strong acid or a strong base is added resulting in 0.1 mM increase of the acid or the base in the solution.

Conversely, suitably, a substance is not considered to be a buffer if it is not capable of reducing the magnitude of the pH change of a solution to 75%, preferably 50%, most preferably to 25%, compared with an identical solution that does not comprise the substance, when either strong acid or a strong base is added resulting in 0.1 mM increase of the acid or the base in the solution.

In one embodiment, the or a buffer is an amino acid. In another embodiment, the or a buffer is not an amino acid.

Where present, suitable buffers include, but are not limited to: histidine, maleate, sulphite, glyoxylate, aspartame, glucuronate, aspartate, glutamate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane, N-tris(hydroxymethyl)glycine and N-tris(hydroxymethyl)methyl-3-aminopropanesulphonic acid, and salts thereof, and combinations thereof. In one embodiment, the buffer is selected from the group consisting of histidine, maleate, tartrate, lactate, benzoate, acetate, bicarbonate, phosphate and tris(hydroxymethyl)aminomethane, in particular histidine, lactate, acetate, phosphate and tris(hydroxymethyl)aminomethane. In one embodiment, the buffer is citrate.

In one embodiment, the composition further comprises species selected from the group consisting of gentisate and salicylate. These species may for example be present at a concentration of up to 5 mM. In an embodiment these species are absent from the composition.

In one embodiment, the composition is free of malic acid.

In one embodiment, the composition solvent comprises (based on volume) at least 80% water, such as at least 85% water, at least 90% water, at least 95% water or at least 99% water. In one embodiment, the solvent is water i.e. the only solvent present in the composition is water. Suitably, the composition is free of ethanol.

As used herein, the term “peptide therapeutic agent” refers to a peptide which does not contain ionisable groups with pk a in the range 3.0 to 8.5. As such, the peptide therapeutic agent will not provide any substantial buffering capacity in the pH range 4.0-7.5, i.e. is not capable of “self-buffering”.

Typically, a peptide therapeutic agent is considered to provide substantial buffering capacity if it is capable of reducing the magnitude of the pH change of a solution to 75%, preferably 50%, most preferably to 25%, compared with an identical solution that does not comprise the peptide therapeutic agent, when either strong acid or a strong base is added resulting in 0.1 mM increase of the acid or the base in the solution.

Conversely, typically, a peptide therapeutic agent is considered not to provide any substantial buffering capacity if it is not capable of reducing the magnitude of the pH change of a solution to 75%, preferably 50%, most preferably to 25%, compared with an identical solution that does not comprise the peptide therapeutic agent, when either strong acid or a strong base is added resulting in 0.1 mM increase of the acid or the base in the solution.

In one embodiment is provided an aqueous solution composition of pH in the range 4.0 to 7.5 comprising:
a peptide therapeutic agent;
optionally one or more buffers being substances having at least one ionisable group with a pkₐ in the range 3.0 to 8.5 and which pkₐ is within 2 pH units of the pH of the composition; and
a stabilizer;
   wherein the buffers are present in the composition at a total concentration of 0-5 mM, and wherein the peptide therapeutic agent is not capable of reducing the magnitude of the pH change of a solution to 75%, preferably 50%, most preferably to 25%, compared with an identical solution that does not comprise the peptide therapeutic agent, when either strong acid or a strong base is added resulting in 0.1 mM increase of the acid or the base in the solution.

In one embodiment is provided an aqueous solution composition of pH in the range 4.0 to 7.5 comprising:
a peptide therapeutic agent;
optionally one or more buffers being substances having at least one ionisable group with a pkₐ in the range 3.0 to 8.5 and which pkₐ is within 2 pH units of the pH of the composition; and
a stabilizer;
   wherein the buffers are present in the composition at a total concentration of 0-5 mM, wherein the peptide therapeutic agent does not contain ionisable groups with pkₐ in the range 3.0 to 8.5, and wherein the peptide therapeutic agent is not capable of reducing the magnitude of the pH change of a solution to 75%, preferably 50%, most preferably to 25%, compared with an identical solution that does not comprise the peptide therapeutic agent, when either strong acid or a strong base is added resulting in 0.1 mM increase of the acid or the base in the solution.

In an embodiment, the peptide therapeutic agent does not contain ionisable groups with pk a in the range 2.0 to 9.5.

In one embodiment, the peptide therapeutic agent does not contain any free aspartic acid, free glutamic acid, histidine or free cysteine side chains, these side chains all having pk a values between 3.0 and 8.5. By “free” is meant aspartic acid and glutamic acid side chains containing intact CO2H or COO— side chain functionality (i.e. not engaged in an amide or other bond) and cysteine side chains containing intact SH or S-side chain functionality (i.e. not engaged in a disulphide or other bond).

Peptide therapeutic agents, particularly those that are relatively small, may comprise a relatively high amount of a non-pharmacologically active counterion to balance a charge. In the case of positively charged molecules a counter-anion (e.g. chloride or acetate) is needed, whereas a counter-cation (e.g. sodium cation) is needed in the case of negatively charged molecules. The counterion (e.g. acetate) may have a buffering capacity itself. However, when considering whether or not the peptide therapeutic agent provides any buffering capacity in the pH range 4.0-7.5, any such counter-ions should be excluded from the assessment. Thus, a peptide therapeutic agent may be associated with a non-pharmacologically active counter-ion which is capable of buffering in the pH range 4.0-7.5, but provided the peptide therapeutic agent itself is not, according to the present invention such a peptide therapeutic agent does not provide any buffering capacity in the range pH 4.0-7.5. However, when calculating the concentration of buffer in the aqueous solution composition, any such counterions with buffering capacity should be included in the total concentration of buffer.

The peptide therapeutic agent is not a protein. By “protein” is meant a chain of amino acids that is of sufficient length (exceeding 25 amino acids in length) to produce higher levels of secondary and/or tertiary and/or quaternary structure. A “peptide” as defined herein is a short chain of up to 25, such as up to 20, up to 15, up to 12 or up to 10 amino acid monomers, linked by peptide bonds.

Thus, in one embodiment, the peptide therapeutic agent contains up to 25, such as up to 20, up to 15, up to 12 or up to 10 amino acids. The peptide therapeutic agent may be unbranched, branched, linear, cyclic and/or partially cyclic. A peptide therapeutic agent may be composed of one or more chains of amino acids linked by one or more suitable linkers (e.g. by a disulphide bond), provided the total number of amino acids does not exceed 25 or 20, or 15, or 12 or 10. The amino acids of the peptide therapeutic agent may include amino acids selected from the 20 natural (proteogenic) amino acids, their derivatives, and other α-amino acids, including non-natural amino acids. Derivatives of natural amino acids include derivatives in which carboxylate groups are converted to an ester or amide such as a C1-6 alkyl ester such as a methyl or ethyl ester or an amide such as a primary amide (i.e. the amide formed by condensation with ammonia), secondary amide (e.g. the amide formed by condensation with methylamine) or tertiary amide (e.g. the amide formed by condensation with dimethylamine), particularly a primary amide; or in which amine groups are converted to an amide e.g. as —NH(CO)C1-5 alkyl such as acetamide. Suitably, the peptide therapeutic agent comprises amino acids which are exclusively selected from the 20 natural (proteogenic) amino acids and their derivatives (for example selected from the 20 natural (proteogenic) amino acids).

A peptide therapeutic agent may have a free amine (—NH2) group at the N-terminus. Alternatively, the N-terminal amine group may be derivatized e.g. as an amide group (e.g. —NH(CO)C1-20 alkyl such as —NH(CO)C1-5 alkyl, particularly as acetamide). A peptide therapeutic agent may have a free carboxylic acid (—COOH) group at the C-terminus. Alternatively, the C-terminal carboxylic acid group may be derivatized e.g. as an amide group (e.g. a primary amide (i.e. the amide formed by condensation with ammonia), secondary amide (e.g. the amide formed by condensation with a primary amine such as methylamine) or tertiary amide (e.g. the amide formed by condensation with a secondary amine such as dimethylamine), particularly a primary amide) or as an ester group (e.g. a C1-6 alkyl ester such as a methyl or ethyl ester).

Alternatively, the peptide therapeutic agent may be cyclic whereby the N and C termini are joined, either directly or via a linker. Alternatively, the peptide therapeutic agent may be partially cyclic whereby the N- or C-terminus is joined to an amino acid side chain, either directly or via a linker. Amino acid side chains that may join to the N or C terminus include those deriving from cysteine, glutamic acid and aspartic acid. Linkers may, for example, be chains of 2-10 atoms in length which atoms are selected from carbon, nitrogen, sulphur and oxygen atoms. Linkers may comprise functionalised atoms (e.g. atoms substituted by hydroxy, amino or oxo) and/or bear carbon containing side chains.

Cyclic and partially cyclic peptide therapeutic agents include those involving an intramolecular disulphide bond e.g. between two cysteine side chains.

In a preferred embodiment, the peptide therapeutic agent does not have a free N-terminal amine group or does not have a free C-terminal carboxylic acid group and preferably has neither a free N-terminal amine group nor a free C-terminal carboxylic acid group, for example because the peptide therapeutic agent is cyclic or partially cyclic and/or because the N-terminus is derivatized (e.g. as an amide) and/or the C-terminus is derivatized (e.g. as an ester or an amide).

The aqueous composition of the invention may be of use for any peptide therapeutic agent of interest, provided the peptide therapeutic agent does not contain ionisable groups with pK a in the range of 3.0 to 8.5, and suitably does not comprise free aspartic acid, free glutamic acid, histidine or free cysteine side chains.

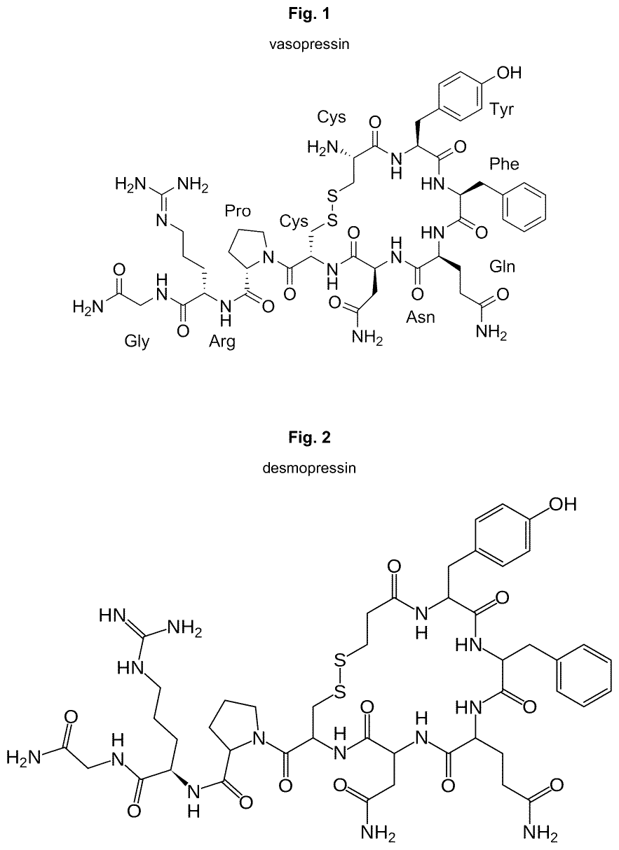

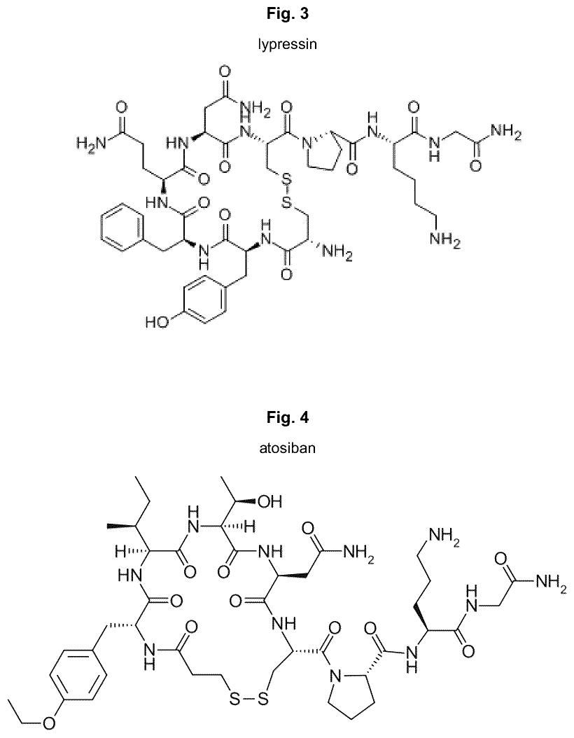

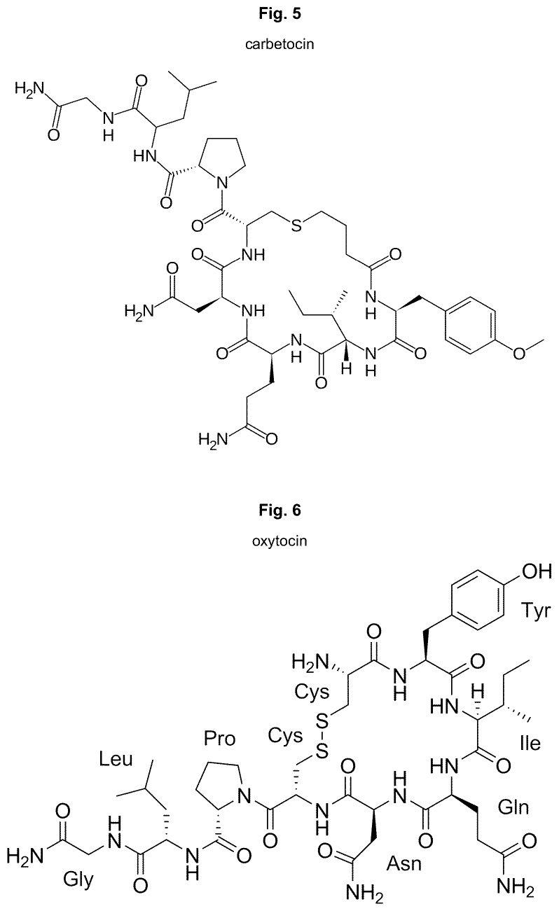

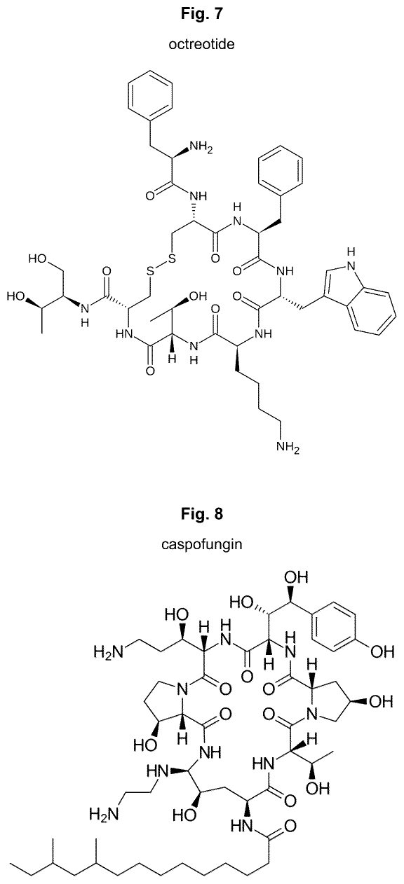

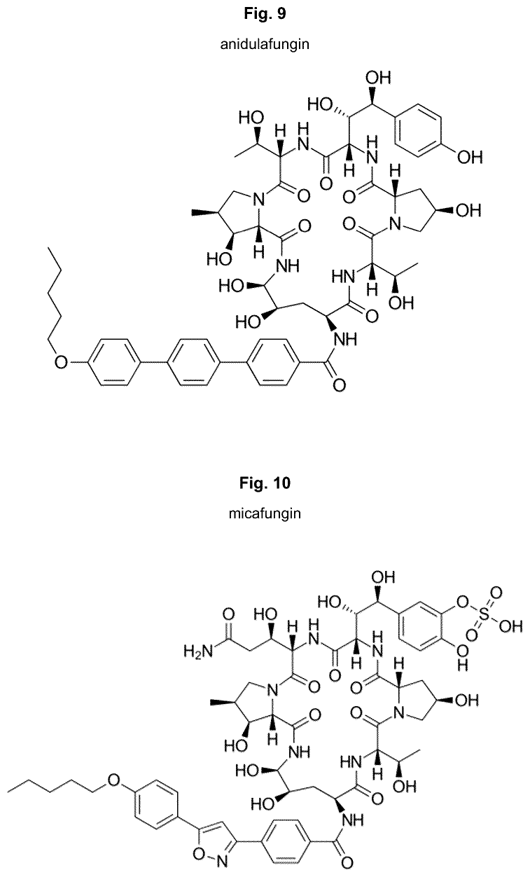

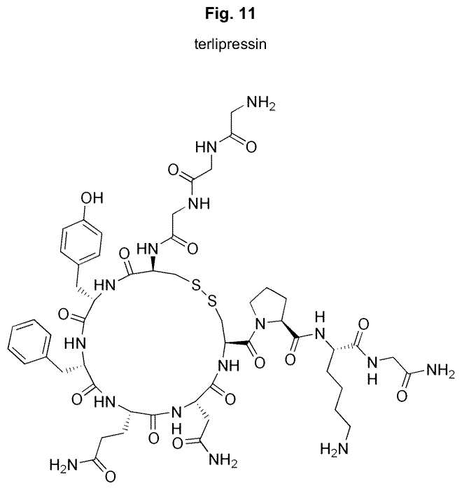

Peptide therapeutic agents may be naturally occurring or non-naturally occurring. Examples of peptide therapeutic agents which do not have ionisable groups with pk a in the range of 3.0 to 8.5 are vasopressin and analogues thereof. In one embodiment, the peptide therapeutic agent is vasopressin (also known as arginine vasopressin, see FIG. 1). In one embodiment, the peptide therapeutic agent is desmopressin (see FIG. 2). In one embodiment, the peptide therapeutic agent is lypressin (also known as lysine vasopressin, see FIG. 3). Other suitable peptide therapeutic agents include atosiban (see FIG. 4), carbetocin (see FIG. 5), oxytocin (see FIG. 6), octreotide (see FIG. 7), caspofungin (see FIG. 8), anidulafungin (see FIG. 9) and micafungin (see FIG. 10). In one embodiment, the peptide therapeutic agent is terlipressin (See FIG. 11). It will be understood that such peptide therapeutic agents may be employed in the formulation in the form of any of their pharmaceutically acceptable salts.

The following specific substances are not peptide therapeutic agents covered by the invention as defined herein: insulin, an insulin analogue such as insulin lispro, insulin aspart, insulin glulisine or insulin glargine, glucagon, human grown hormone, gonadotropin, human thyroid stimulation hormone, granulocyte colony stimulation hormone, streptokinase, asparaginase, urate oxidase, antigens of vaccines such as hepatitis B vaccine, malaria vaccine, human papilloma vaccine, meningitis A vaccine, meningitis C vaccine, pertussis vaccine and polio vaccine, antibodies such as anti-epidermal growth factor receptor (EGFR) monoclonal antibody, anti-HER2 monoclonal antibody, anti-CD 52 monoclonal antibody and anti-CD 20 monoclonal antibody, polyclonal antibodies, interferons, erythropoietin, darbopoietin alpha, blood coagulation factors such as Factor VIII and Factor IX human albumin, protein C, hormones generally, microbial proteins, metabolic proteins, soluble forms of structural proteins and the direct thrombin inhibitor bivalirudin. Said substances are excluded because they are proteins (i.e. are not peptides) and/or because they contain ionisable groups, in particular ionisable amino acid sidechains, with pk a values in the range 3.0 to 8.5.

The concentration of peptide therapeutic agent in the composition is typically 0.001-50 mg/ml, such as 0.01-10 mg/ml or 0.1-5 mg/ml.

The present inventors have discovered that as well as minimising buffer concentration, the addition of a stabilizer to the composition can provide further stability benefits. A stabilizer will be employed in a stabilizing amount.

In one embodiment, the stabilizer is selected from amino acids, particularly natural amino acids, such as α-amino acids. Amino acids that have side chains with pk a in the range 3.0 to 8.5 are not suitable as stabilizers. In one embodiment, the stabilizer is an amino acid selected from methionine, arginine, glycine and proline, in particular selected from methionine, arginine and proline, or is methionine. The amino acid as stabilizer is present in a concentration sufficient to provide a stabilizing effect. In one embodiment, the amino acid as stabilizer is present at a concentration of 1-200 mM, such as 10-100 mM or 10-50 mM.

In another embodiment, the stabilizer is a polyol, suitably selected from the group consisting of glycerol, mannitol, propylene glycol, PEG 300, PEG 400, sucrose, trehalose and lactose. Preferred polyol stabilizers are selected from glycerol, mannitol, propylene glycol and sucrose. The polyol as stabilizer is present in a concentration sufficient to provide a stabilizing effect. In one embodiment, the polyol as stabilizer is present at a concentration of 1-1000 mM, such as 10-500 mM or 100-500 mM. When the stabilizer is a polyol it may also serve as tonicity modifier. In some embodiments, the stabilizer is a polyol but an additional tonicity modifier (i.e. in addition to the polyol) is included in the composition. Thus, in one embodiment, the composition comprises a polyol as stabilizer (suitably glycerol, mannitol, propylene glycol or sucrose) and an additional tonicity modifier. The additional tonicity modifier can be either charged or uncharged, e.g. as defined below.

The pH of the composition is between 4.0 and 7.5, suitably between 4.0 and 7.0 or between 4.0 and 6.0.

The composition may comprise a tonicity modifier, which may be charged or uncharged. Examples of uncharged tonicity modifiers include glycerol, 1,2-propanediol, mannitol, sorbitol, trehalose, PEG 300 and PEG400. When included, an uncharged tonicity modifier is typically employed in the composition at a concentration of 50-1000 mM, for example 200-500 mM, such as about 300 mM. Examples of charged tonicity modifiers include sodium chloride, sodium sulphate, and amino acids such as glycine or arginine. Amino acids that have side chains with pk a in the range 3.0 to 8.5 are not suitable as tonicity modifiers. When included, a charged tonicity modifier is typically employed in the composition at a concentration of 25-500 mM, for example 50-250 mM such as about 150 mM. An uncharged tonicity modifier rather than a charged tonicity modifier is generally preferred.

The composition may comprise a non-ionic surfactant.

A particularly suitable class of non-ionic surfactants is the polysorbates (fatty acid esters of ethoxylated sorbitan), such as polysorbate 20 or polysorbate 80. Polysorbate 20 is a mono ester formed from lauric acid and polyoxyethylene (20) sorbitan in which the number 20 indicates the number of oxyethylene groups in the molecule. Polysorbate 80 is a mono ester formed from oleic acid and polyoxyethylene (20) sorbitan in which the number 20 indicates the number of oxyethylene groups in the molecule. Polysorbate 20 is known under a range of brand names including in particular Tween 20, and also Alkest TW 20. Polysorbate 80 is known under a range of brand names including in particular Tween 80, and also Alkest TW 80. Other suitable polysorbates include polysorbate 40 and polysorbate 60.

Another suitable class of non-ionic surfactants is the alkyl glycosides, especially dodecyl maltoside. Other alkyl glycosides include dodecyl glucoside, octyl glucoside, octyl maltoside, decyl glucoside, decyl maltoside, tridecyl glucoside, tridecyl maltoside, tetradecyl glucoside, tetradecyl maltoside, hexadecyl glucoside, hexadecyl maltoside, sucrose monooctanoate, sucrose mono decanoate, sucrose monododecanoate, sucrose monotridecanoate, sucrose monotetradecanoate and sucrose monohexadecanoate.

Another suitable class of non-ionic surfactants is block copolymers of polyethylene glycol and polypropylene glycol, also known as poloxamers, especially poloxamer 188, poloxamer 407, poloxamer 171 and poloxamer 185. Poloxamers are also known under brand names Pluronics or Koliphors. For example, poloxamer 188 is marketed as Pluronic F-68.

Another suitable class of non-ionic surfactants is alkyl ethers of polyethylene glycol, especially those known under a brand name Brij, such as selected from polyethylene glycol (2) hexadecyl ether (Brij 52), polyethylene glycol (2) oleyl ether (Brij 93) and polyethylene glycol (2) dodecyl ether (Brij L4). Other suitable Brij surfactants include polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

Another suitable class of non-ionic surfactants are alkylphenyl ethers of polyethylene glycol, especially 4-(1,1,3,3-tetramethylbutyl)phenyl-polyethylene glycol, also known under a brand name Triton X-100.

In one embodiment, the non-ionic surfactant is a polysorbate or a poloxamer, and is suitably a polysorbate. The concentration of the non-ionic surfactant in the composition will typically be in the range 10-2000 μg/ml, such as 50-1000 μg/ml, 100-500 μg/ml or about 200 μg/ml.

In one embodiment, the non-ionic surfactant is selected from the group consisting of an alkyl glycoside, a polysorbate, an alkyl ether of polyethylene glycol, a block copolymer of polyethylene glycol and polypropylene glycol, and an alkylphenyl ether of polyethylene glycol.

The compositions of the invention may additionally comprise a preservative such as a phenolic or a benzylic preservative. The preservative is suitably selected from the group consisting of phenol, m-cresol, chlorocresol, benzyl alcohol, propyl paraben and methyl paraben, in particular phenol, m-cresol and benzyl alcohol. The concentration of preservative is typically 10-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

In one embodiment, there is provided an aqueous solution composition of pH in the range 4.0 to 7.5 consisting of:
a peptide therapeutic agent;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the peptide therapeutic agent does not contain ionisable groups with pk a in the range 3.0 to 8.5, and wherein the buffers are present in the composition at a total concentration of 0-5 mM.

In one embodiment, there is provided an aqueous solution composition of pH in the range 4.0 to 7.5 consisting of:
a peptide therapeutic agent;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the peptide therapeutic agent does not contain ionisable groups with pk a in the range 3.0 to 8.5, wherein the buffers are present in the composition at a total concentration of 0-5 mM, such as 0.5 to 5 mM, and wherein the stabilizer is an amino acid such as methionine, arginine or proline (in particular methionine) or a polyol such as glycerol, mannitol, propylene glycol or sucrose.

In one embodiment, there is provided an aqueous solution composition of pH in the range 4.0 to 7.5 consisting of:
a peptide therapeutic agent;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the peptide therapeutic agent does not contain any free aspartic acid, free glutamic acid, histidine or free cysteine side chains, wherein the buffers are present in the composition at a total concentration of 0-5 mM, such as 0.5-5 mM, and wherein the stabilizer is an amino acid such as methionine, arginine or proline (in particular methionine), or a polyol such as glycerol, mannitol, propylene glycol or sucrose.

In one embodiment, there is provided an aqueous solution composition of pH in the range 4.0 to 7.5 consisting of:
a peptide therapeutic agent;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the peptide therapeutic agent does not contain ionisable side chains with pk a in the range of 3.0 to 8.5, such as side chains of free aspartic acid, free glutamic acid, histidine or free cysteine, wherein the buffers are present in the composition at a total concentration of 0-5 mM, such as 0.5-5 mM, and wherein the stabilizer is an amino acid such as methionine, arginine or proline (in particular methionine), or a polyol such as glycerol, mannitol, propylene glycol or sucrose.

In one embodiment, there is provided an aqueous solution composition of pH in the range 4.0 to 7.5 consisting of:
a peptide therapeutic agent;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the peptide therapeutic agent is selected from the group consisting of vasopressin, lypressin, desmopressin, atosiban, carbetocin, oxytocin, octreotide, caspofungin, anidulafungin and micafungin, wherein the buffers are present in the composition at a total concentration of 0-5 mM, such as 0.5 to 5 mM, and wherein the stabilizer is an amino acid such as methionine, arginine or proline (in particular methionine), or a polyol such as glycerol, mannitol, propylene glycol or sucrose.

In one embodiment, there is provided an aqueous solution composition of pH in the range 4.0 to 7.5 consisting of:
a peptide therapeutic agent;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the peptide therapeutic agent is selected from the group consisting of vasopressin, lypressin, desmopressin, atosiban, carbetocin, oxytocin, octreotide, caspofungin, anidulafungin, micafungin and terlipressin, wherein the buffers are present in the composition at a total concentration of 0-5 mM, such as 0.5 to 5 mM, and wherein the stabilizer is an amino acid such as methionine, arginine or proline (in particular methionine), or a polyol such as glycerol, mannitol, propylene glycol or sucrose.

In one embodiment, there is provided an aqueous solution composition of pH in the range 4.0 to 7.5 consisting of:
a peptide therapeutic agent;
optionally one or more buffers being substances having at least one ionisable group with a pkₐ in the range 3.0 to 8.5 and which pKₐ is within 1 pH unit of the pH of the composition;
a stabilizer;
optionally one or more preservatives;
optionally one or more non-ionic surfactants; and
optionally one or more tonicity modifiers;
   wherein the peptide therapeutic agent does not contain ionisable groups with pkₐ in the range 3.0 to 8.5, wherein the buffers are present in the composition at a total concentration of 0-5 mM, and wherein the stabilizer is an amino acid such as methionine, arginine or proline (in particular methionine) or a polyol such as glycerol, mannitol, propylene glycol or sucrose.

In one embodiment, there is provided an aqueous solution composition of pH in the range 4.0-7.5 comprising:
a peptide therapeutic agent selected from the group consisting of vasopressin, lypressin, desmopressin, atosiban, carbetocin, oxytocin, octreotide, caspofungin, anidulafungin and micafungin;
optionally one or more buffers being substances having at least one ionisable group with a pkₐ in the range 3.0 to 8.5 and which pkₐ is within 2 pH units of the pH of the composition; and
a stabilizer;
   wherein the buffers are present in the composition at a total concentration of 0-5 mM.

In one embodiment, there is provided an aqueous solution composition of pH in the range 4.0-7.5 comprising:
a peptide therapeutic agent selected from the group consisting of vasopressin, lypressin, desmopressin, atosiban, carbetocin, oxytocin, octreotide, caspofungin, anidulafungin, micafungin and terlipressin;
optionally one or more buffers being substances having at least one ionisable group with a pkₐ in the range 3.0 to 8.5 and which pKₐ is within 2 pH units of the pH of the composition; and
a stabilizer;
   wherein the buffers are present in the composition at a total concentration of 0-5 mM.

In one embodiment, there is provided an aqueous solution composition of pH in the range 4.0-7.5 consisting of:
a peptide therapeutic agent selected from the group consisting of vasopressin, lypressin, desmopressin, atosiban, carbetocin, oxytocin, octreotide, caspofungin, anidulafungin and micafungin;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the buffers are present in the composition at a total concentration of 0-5 mM.

In one embodiment, there is provided an aqueous solution composition of pH in the range 4.0-7.5 consisting of:
a peptide therapeutic agent selected from the group consisting of vasopressin, lypressin, desmopressin, atosiban, carbetocin, oxytocin, octreotide, caspofungin, anidulafungin, micafungin and terlipressin;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the buffers are present in the composition at a total concentration of 0-5 mM.

In one embodiment, there is provided an aqueous solution composition of pH in the range 4.0-7.5 consisting of:
a peptide therapeutic agent selected from the group consisting of vasopressin, lypressin, desmopressin, atosiban, carbetocin, oxytocin, octreotide, caspofungin, anidulafungin and micafungin;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the buffers are present in the composition at a total concentration of 0-5 mM, and wherein the stabilizer is an amino acid such as methionine, arginine or proline (in particular methionine) or a polyol such as glycerol, mannitol, propylene glycol or sucrose.

In one embodiment, there is provided an aqueous solution composition of pH in the range 4.0-7.5 consisting of:
a peptide therapeutic agent selected from the group consisting of vasopressin, lypressin, desmopressin, atosiban, carbetocin, oxytocin, octreotide, caspofungin, anidulafungin, micafungin and terlipressin;
optionally one or more buffers being substances having at least one ionisable group with a pkₐ in the range 3.0 to 8.5 and which pkₐ is within 2 pH units of the pH of the composition;
a stabilizer;
optionally one or more preservatives;
optionally one or more non-ionic surfactants; and
optionally one or more tonicity modifiers;
   wherein the buffers are present in the composition at a total concentration of 0-5 mM, and wherein the stabilizer is an amino acid such as methionine, arginine or proline (in particular methionine) or a polyol such as glycerol, mannitol, propylene glycol or sucrose.

In one embodiment, there is provided an aqueous solution composition of pH in the range 4.0-7.5 comprising:
a peptide therapeutic agent;
optionally one or more buffers being substances having at least one ionisable group with a pkₐ in the range 3.0 to 8.5 and which pkₐ is within 2 pH units of the pH of the composition;
a stabilizer which is an amino acid selected from methionine, arginine and proline; and
an uncharged tonicity modifier selected from glycerol, 1,2-propanediol, mannitol, sorbitol, trehalose, PEG 300 and PEG 400;
   wherein the peptide therapeutic agent does not contain ionisable groups with pkₐ in the range 3.0 to 8.5, and wherein the buffers are present in the composition at a total concentration of 0-5 mM.

In one embodiment, there is provided an aqueous solution composition of pH in the range 4.0-7.5 consisting of:
a peptide therapeutic agent;
optionally one or more buffers being substances having at least one ionisable group with a pkₐ in the range 3.0 to 8.5 and which pkₐ is within 2 pH units of the pH of the composition;
a stabilizer which is an amino acid selected from methionine, arginine and proline; and
an uncharged tonicity modifier selected from glycerol, 1,2-propanediol, mannitol, sorbitol, trehalose, PEG300 and PEG400;
   wherein the peptide therapeutic agent does not contain ionisable groups with pkₐ in the range 3.0 to 8.5, and wherein the buffers are present in the composition at a total concentration of 0-5 mM.

In one embodiment, there is provided an aqueous solution composition of pH in the range 4.0-7.5 comprising:
a peptide therapeutic agent;
optionally one or more buffers being substances having at least one ionisable group with a pkₐ in the range 3.0 to 8.5 and which pkₐ is within 2 pH units of the pH of the composition;
a stabilizer which is methionine; and
an uncharged tonicity modifier which is mannitol;
   wherein the peptide therapeutic agent does not contain ionisable groups with pkₐ in the range 3.0 to 8.5, and wherein the buffers are present in the composition at a total concentration of 0-5 mM.

In one embodiment, there is provided an aqueous solution composition of pH in the range 4.0-7.5 consisting of:
a peptide therapeutic agent;
optionally one or more buffers being substances having at least one ionisable group with a pkₐ in the range 3.0 to 8.5 and which pkₐ is within 2 pH units of the pH of the composition;
a stabilizer which is methionine; and
an uncharged tonicity modifier which is mannitol;
   wherein the peptide therapeutic agent does not contain ionisable groups with pkₐ in the range 3.0 to 8.5, and wherein the buffers are present in the composition at a total concentration of 0-5 mM.

The presently claimed invention derives from the surprising observation that compositions comprising a peptide therapeutic agent are stabilized at low buffer concentrations. Such solutions may be further stabilized by the addition of a stabilizer such as an amino acid or a polyol. The solution may be even further stabilized by the addition of an uncharged tonicity modifier.

Suitably the composition of the invention remains as a clear solution following storage at 2-8° C. for extended period of time, such as at least 12 months, preferably 18 months, most preferably at least 24 months.

Suitably the composition of the invention remains as a clear solution following storage at 25° C. for extended period of time, such as at least 6 months, preferably at least 12 months, such as at least 18 months, such as at least 24 months.

Suitably the composition of the invention remains as a clear solution following storage at 30° C. for extended period of time, such as at least 6 months, preferably at least 12 months, such as at least 18 months, such as at least 24 months.

Suitably the composition of the invention has improved storage stability either at 2-8° C. or at increased temperature than in an equivalent composition that comprises higher concentration of the same buffer or buffers.

Suitably the composition of the invention has improved storage stability either at 2-8° C. or at increased temperature than in an equivalent composition that does not comprise the stabilizer.

In one embodiment, the composition of the invention comprises no more than 5% total impurities, such as no more than 4%, such as no more than 3%, such as no more than 2% total impurities (by total weight of peptide therapeutic agent in the composition, as measured by RP-HPLC (Reversed-Phase High-Performance Liquid Chromatography) or a similar suitable technique) following storage at 2-8° C. for at least 12 months, preferably 18 months, most preferably 24 months.

In one embodiment, the composition of the invention comprises no more than 5% total impurities, such as no more than 4%, such as no more than 3%, such as no more than 2% total impurities (by total weight of peptide therapeutic agent in the composition, as measured by RP-HPLC or a similar suitable technique) following storage at 25° C. for at least 6 months, preferably 12 months, such as 18 months or 24 months.

In one embodiment, the composition of the invention comprises no more than 5% total impurities, such as no more than 4%, such as no more than 3%, such as no more than 2% total impurities (by total weight of peptide therapeutic agent in the composition, as measured by RP-HPLC or a similar suitable technique) following storage at 30° C. for at least 6 months, preferably 12 months, such as 18 months or 24 months.

In one embodiment, the composition of the invention comprises no more than 5% total impurities, such as no more than 4%, such as no more than 3%, such as no more than 2% total impurities (by total weight of peptide therapeutic agent in the composition, as measured by RP-HPLC or a similar suitable technique) following storage at 40° C. for at least 2 weeks, preferably 4 weeks, such as 6 weeks.

In one embodiment, the composition of the invention comprises lower level of impurities (as measured by RP-HPLC or a similar suitable technique) than a commercially available composition comprising the same pharmaceutical ingredient (as measured by RP-HPLC or a similar suitable technique) following storage at 2-8° C. for at least 12 months, preferably 18 months, most preferably 24 months.

In one embodiment, the composition of the invention comprises lower level of impurities (as measured by RP-HPLC or a similar suitable technique) than a commercially available composition comprising the same pharmaceutical ingredient (as measured by RP-HPLC or a similar suitable technique) following storage at 25° C. for at least 6 months, preferably 12 months, such as 18 months or 24 months.

In one embodiment, the composition of the invention comprises lower level of impurities (as measured by RP-HPLC or a similar suitable technique) than a commercially available composition comprising the same pharmaceutical ingredient (as measured by RP-HPLC or a similar suitable technique) following storage at 30° C. for at least 6 months, preferably 12 months, such as 18 months or 24 months.

In a further aspect of the invention, there is provided a method of improving the stability of an aqueous solution composition of pH in the range of 4.0 to 7.5 comprising a peptide therapeutic agent, which comprises adding to the composition a stabilizer and maintaining a low buffer concentration, wherein the peptide therapeutic agent does not contain ionisable groups with pK a in the range 3.0 to 8.5, and wherein the buffer is present in the composition at a concentration of 0-5 mM.

In a further aspect of the invention, there is provided the use of a stabilizer for improving the stability of an aqueous solution composition of pH in the range of 4.0 to 7.5 comprising a peptide therapeutic agent, wherein the peptide therapeutic agent does not contain ionisable groups with pk a in the range 3.0 to 8.5, and wherein the buffer or buffers being substances having at least one ionisable group with a pk a in the range 3.0 to 8.5 and which pk a is within 2 pH units of the pH of the composition are present in the composition at a total concentration of 0-5 mM.

In a further aspect of the invention, there is provided the use of a minimal amount of buffer for improving the stability of an aqueous solution composition of pH in the range of 4.0 to 7.5 comprising a peptide therapeutic agent, wherein the peptide therapeutic agent does not contain ionisable groups with pkₐ in the range 3.0 to 8.5, wherein the composition comprises a stabilizer, and wherein the buffer or buffers being substances having at least one ionisable group with a pkₐ in the range 3.0 to 8.5 and which pkₐ is within 2 pH units of the pH of the composition are present in the composition at a total concentration of 0-5 mM.

In an embodiment, the composition of the invention is a composition for use in therapy. In an embodiment, the composition of the invention is a pharmaceutical composition.

In one embodiment is provided an aqueous solution composition of pH in the range 4.0-7.5 for use in therapy comprising:
a peptide therapeutic agent;
optionally one or more buffers being substances having at least one ionisable group with a pkₐ in the range 3.0 to 8.5 and which pkₐ is within 2 pH units of the pH of the composition; and
a stabilizer;
   wherein the peptide therapeutic agent does not contain ionisable groups with pkₐ in the range 3.0 to 8.5, and wherein the buffers are present in the composition at a total concentration of 0-5 mM.

In one embodiment is provided an aqueous solution composition which is a pharmaceutical composition, of pH in the range 4.0-7.5 comprising:
a peptide therapeutic agent;
optionally one or more buffers being substances having at least one ionisable group with a pkₐ in the range 3.0 to 8.5 and which pkₐ is within 2 pH units of the pH of the composition; and
a stabilizer;
   wherein the peptide therapeutic agent does not contain ionisable groups with pkₐ in the range 3.0 to 8.5, wherein the buffers are present in the composition at a total concentration of 0-5 mM.

All embodiments described above with respect to the aqueous solution composition apply equally to methods and uses of the invention.

There is also provided a container, for example made of plastics or glass, containing one dose or a plurality of doses of the composition as described herein. The container can be for example, a vial, a pre-filled syringe, a pre-filled infusion bag, or a cartridge designed to be a replaceable item for use with an injection device.

The compositions of the invention may suitably be packaged for injection, especially intravenous infusion, intravenous injection, subcutaneous injection or intramuscular injection.

An aspect of the invention is an injection device, particularly a device adapted for subcutaneous or intramuscular injection, for single or multiple use comprising a container containing one dose or a plurality of doses of the composition of the invention together with an injection needle. In an embodiment, the container is a replaceable cartridge which contains a plurality of doses. In an embodiment, the needle is replaceable e.g. after each occasion of use. In one embodiment, the injection device is in the form of a pen.

Compositions according to the invention are expected to have good physical and chemical stability as described herein.

### EXAMPLES

### General Methods

### Reversed-Phase Chromatography (RP-HPLC)

High performance reverse phase chromatography was performed using the Waters ACQUITY H-class Bio UPLC® system with a 1.7 μm Ethylene Bridged Hybrid particle, 130 Å pore resin trifunctionally immobilised with a C18 ligand in a 50 mm by 15 2.1 mm column. Mobile Phase A was 0.1 M Na₃PO₄ adjusted to pH 3.0 using trifluoroacetic acid. Mobile Phase B was prepared by mixing 2 parts (v/v) of acetonitrile with 1 part (v/v) of Mobile Phase A. The sample comprising a formulated peptide was bound in Mobile Phase A and eluted using a gradient of Mobile Phase A and Mobile Phase B. The sample volume was 10 μl, the flow rate was 0.4 mL/min, with 214 nm UV detection. All analyses were performed at 60° C.

### Example 1: Desmopressin

The effect of acetic acid buffer concentration on the rate of impurity formation in compositions of desmopressin was investigated using the RP-HPLC method described in General Methods, following storage at 40° C. The effect was investigated in the presence of mannitol (300 mM). It was shown that increasing the buffer concentration resulted in stability impairment (comparing compositions 1-6). It was also shown that addition of methionine (5 mM), using 1 mM acetic acid, resulted in improvement of stability (comparing compositions 2 and 7), whilst removing mannitol resulted in stability impairment (comparing compositions 2 and 8). Replacing mannitol with sorbitol did not appear to affect the stability (comparing compositions 2 and 9). The best results at 1 mM buffer concentration were achieved by combining mannitol and methionine.

TABLE 1
Increase in impurity level in compositions of desmopressin
following storage at 40° C. for 4 weeks and 6 weeks.

| | | | | | | Increase | Increase |
|---|---|---|---|---|---|---|---|
| | | | | | | in % | in % |
| | Acetic | Man- | Methi- | Sor- | | impurity | impurity |
| Comp. | acid | nitol | onine | bitol | | (4 weeks | (6 weeks |
| No. | (mM) | (mM) | (mM) | (mM) | pH | storage) | storage) |
| 1 | 0 | 300 | — | — | 4.5 | 0.04 | 0.05 |
| 2 | 1 | 300 | — | — | 4.5 | 0.07 | 0.10 |
| 3 | 3 | 300 | — | — | 4.5 | 0.09 | 0.14 |
| 4 | 5 | 300 | — | — | 4.5 | 0.10 | 0.16 |
| 5 | 20 | 300 | — | — | 4.5 | 0.14 | 0.26 |
| 6 | 50 | 300 | — | — | 4.5 | 0.20 | 0.31 |
| 7 | 1 | 300 | 5 | — | 4.5 | 0.04 | 0.06 |
| 8 | 1 | — | — | — | 4.5 | 0.18 | 0.26 |
| 9 | 1 | — | — | 300 | 4.5 | 0.08 | 0.11 |

### Example 2: A Tosiban

The effect of acetic acid buffer concentration on the rate of impurity formation in compositions of atosiban was investigated using the RP-HPLC method described in General Methods, following storage at 40° C. The effect was investigated in the presence of mannitol (300 mM). It was shown that increasing the buffer concentration resulted in stability impairment (comparing compositions 1-5). It was also shown that addition of proline (5 mM, comparing compositions 2 and 6) or arginine (5 mM, comparing compositions 2 and 7), using 1 mM acetic acid, resulted in further improvement of stability, whilst removing mannitol resulted in stability impairment (comparing compositions 2 and 8).

TABLE 2
Increase in impurity level in compositions of desmopressin
following storage at 40° C. for 4 weeks.

| | Acetic | Man- | | | | Increase |
|---|---|---|---|---|---|---|
| Comp. | acid | nitol | Proline | Arginine | | in % |
| No. | (mM) | (mM) | (mM) | (mM) | pH | impurity |
| 1 | 0 | 300 | — | — | 4.5 | 0.60 |
| 2 | 1 | 300 | — | — | 4.5 | 0.65 |
| 3 | 3 | 300 | — | — | 4.5 | 0.68 |
| 4 | 20 | 300 | — | — | 4.5 | 0.87 |
| 5 | 50 | 300 | — | — | 4.5 | 1.81 |
| 6 | 1 | 300 | 5 | — | 4.5 | 0.55 |
| 7 | 1 | 300 | — | 5 | 4.5 | 0.59 |
| 8 | 1 | — | — | — | 4.5 | 0.91 |

### Example 3: Oxytocin

The effect of methionine, proline, mannitol and sorbitol on the stability of oxytocin in the presence of 1 mM acetic acid buffer (pH 4.5) was investigated using the RP-HPLC method described in General Methods, following storage at 25° C. It was shown that the presence of all of these excipients reduced the rate of impurity formation. The best results were achieved by combining mannitol and methionine.

TABLE 3
Increase in impurity level in compositions of oxytocin
following storage at 25° C. for 4 weeks.

| | Acetic | Methi- | | Man- | Sor- | | Increase |
|---|---|---|---|---|---|---|---|
| Comp. | acid | onine | Proline | nitol | bitol | | in % |
| No. | (mM) | (mM) | (mM) | (mM) | (mM) | pH | impurity |
| 1 | 1 | — | — | — | — | 4.5 | 1.85 |
| 2 | 1 | 5 | — | — | — | 4.5 | 1.03 |
| 3 | 1 | — | 5 | — | — | 4.5 | 1.10 |
| 4 | 1 | — | — | 300 | — | 4.5 | 1.21 |
| 5 | 1 | — | — | — | 300 | 4.5 | 1.06 |
| 6 | 1 | 5 | — | 300 | — | 4.5 | 0.66 |

### Example 4: Caspofungin

The effect of acetic acid buffer concentration on the rate of impurity formation in compositions of caspofungin was investigated using the RP-HPLC method described in General Methods, following storage at 25° C. The effect was investigated in the presence of mannitol (300 mM). It was shown that increasing the buffer concentration resulted in stability impairment (comparing compositions 1-4). It was also shown that removing mannitol resulted in stability impairment. It was also shown that, using 1 mM acetic acid, removal of mannitol resulted in stability impairment (comparing compositions 1-5) whereas replacing mannitol with sorbitol did not appear to have a significant effect of caspofungin stability (comparing compositions 1 and 6).

TABLE 4
Increase in total impurities of oxytocin following
storage of oxytocin at 25° C. for 4 weeks.

| | Acetic | | | | Increase |
|---|---|---|---|---|---|
| Comp. | acid | Mannitol | Sorbitol | | in % of |
| No. | (mM) | (mM) | (mM) | pH | impurities |
| 1 | 1 | 300 | — | 4.5 | 20.5 |
| 2 | 5 | 300 | — | 4.5 | 22.0 |
| 3 | 20 | 300 | — | 4.5 | 28.7 |
| 4 | 50 | 300 | — | 4.5 | 31.3 |
| 5 | 1 | — | — | 4.5 | 31.9 |
| 6 | 1 | — | 300 | 4.5 | 21.6 |

1. An aqueous solution composition of pH in the range 4.0-7.5 comprising:
a peptide therapeutic agent;
optionally one or more buffers being substances having at least one ionisable group with a pk a in the range 3.0 to 8.5 and which pk a is within 2 pH units of the pH of the composition; and
a stabilizer;
wherein the peptide therapeutic agent does not contain ionisable groups with pkₐ in the range 3.0 to 8.5, and wherein the buffers are present in the composition at a total concentration of 0-5 mM.

2. An aqueous solution composition according to claim 1, of pH in the range 4.0-7.5 comprising:
a peptide therapeutic agent;
optionally one or more buffers being substances having at least one ionisable group with a pK a in the range 3.0 to 8.5 and which pk a is within 2 pH units of the pH of the composition;
a stabilizer which is is an amino acid selected from methionine, arginine and proline; and
an uncharged tonicity modifier selected from glycerol, 1,2-propanediol, mannitol, sorbitol, trehalose, PEG300 and PEG400;
wherein the peptide therapeutic agent does not contain ionisable groups with pkₐ in the range 3.0 to 8.5, and wherein the buffers are present in the composition at a total concentration of 0-5 mM.

3. An aqueous solution composition according to claim 1 or claim 2, wherein the peptide therapeutic agent does not contain any free aspartic acid, free glutamic acid, histidine or free cysteine side chains.

4. An aqueous solution composition according to any one of claims 1 to 3, wherein the peptide therapeutic agent does not have a free N-terminal amine group or a free C-terminal carboxylic acid group.

5. An aqueous solution composition according to claim 1 or claim 2, wherein the peptide therapeutic agent is a vasopressin or an analogue such as lypressin or desmopressin or is atosiban, carbetocin, oxytocin, octreotide, caspofungin, anidulafungin or micafungin.

6. An aqueous solution composition according to claim 1 or claim 2, wherein the peptide therapeutic agent is terlipressin.

7. An aqueous solution composition according to any one of claims 1 to 6, wherein the concentration of peptide therapeutic agent in the composition is 0.001-50 mg/ml, such as 0.01-10 mg/ml or 0.1-5 mg/ml.

8. An aqueous solution composition according to any one of claims 1 to 7, wherein the buffer comprises ionisable groups with pk a within 1 unit of the pH of the composition.

9. An aqueous solution composition according to any one of claims 1 to 8, wherein the total concentration of buffers in the composition is 0.1-5 mM, such as 0.1-4 mM, 0.1-3 mM, 0.1-2 mM or 0.1-1 mM.

10. An aqueous solution composition according to any one of claims 1 to 9, wherein the aqueous solution composition is substantially free of buffer.

11. An aqueous solution composition according to any one of claims 1 to 9, wherein the buffer or buffers is/are selected from the group consisting of histidine, maleate, sulphite, glyoxylate, aspartame, glucuronate, aspartate, glutamate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane, N-tris(hydroxymethyl)glycine and N-tris(hydroxymethyl)methyl-3-aminopropanesulphonic acid, and salts thereof, and combinations thereof.

12. An aqueous solution composition according to claim 11, wherein the buffer is selected from the group consisting of histidine, maleate, tartrate, lactate, benzoate, acetate, bicarbonate, phosphate and tris(hydroxymethyl)aminomethane.

13. An aqueous solution composition according to claim 1 or to any one of claims 3 to 12, wherein the stabilizer is an amino acid or a polyol.

14. An aqueous solution composition according to claim 13, wherein the stabilizer is an amino acid selected from methionine, arginine, glycine and proline, in particular methionine.

15. An aqueous solution composition according to claim 14, wherein the stabilizer is an amino acid selected from methionine, arginine and proline.

16. An aqueous solution composition according to claim 13, wherein the stabilizer is a polyol selected from the group consisting of glycerol, mannitol, propylene glycol, PEG 300,PEG 400, sucrose, trehalose and lactose, in particular glycerol, mannitol, propylene glycol and sucrose.

17. An aqueous solution composition according to claim 14, wherein the stabilizer is present at a concentration of 1-200 mM, such as 10-100 mM or 10-50 mM.

18. An aqueous solution composition according to claim 16, wherein the stabilizer is present at a concentration of 1-1000 mM, such as 10-500 mM or 100-500 mM.

19. An aqueous solution composition according to any one of claims 1 to 18, wherein the pH is between 4.0 and 7.0, such as between 4.0 and 6.0.

20. An aqueous solution composition according to any one of claims 1 to 19, further comprising a tonicity modifier.

21. An aqueous solution composition according to claim 20, wherein the tonicity modifier is an uncharged tonicity modifier and is suitably selected from glycerol, 1,2-propanediol, mannitol, sorbitol, trehalose, PEG300 and PEG400.

22. An aqueous solution composition according to claim 21, wherein the tonicity modifier is an uncharged tonicity modifier and is selected from mannitol and sorbitol.

23. An aqueous solution composition according to claim 21 or claim 22, wherein the concentration of the uncharged tonicity modifier is 50-1000 mM, such as 200-500 mM, or about 300 mM.

24. An aqueous solution composition according to claim 20, wherein the tonicity modifier is a charged tonicity modifier and is suitably selected from sodium chloride, sodium sulphate, and an amino acid e.g. glycine or arginine.

25. An aqueous solution composition according to claim 24, wherein the concentration of the charged tonicity modifier is 25-500 mM, such as 50-250 mM, or about 150 mM.

26. An aqueous solution composition according to any one of claims 1 to 25, further comprising a non-ionic surfactant.

27. An aqueous solution composition according to claim 26, wherein the non-ionic surfactant is selected from the group consisting of an alkyl glycoside, a polysorbate, an alkyl ether of polyethylene glycol, a block copolymer of polyethylene glycol and polypropylene glycol, and an alkylphenyl ether of polyethylene glycol.

28. An aqueous solution composition according to claim 27, wherein the non-ionic surfactant is a polysorbate such as polysorbate 20 or polysorbate 80.

29. An aqueous solution composition according to any one of claims 26 to 28, wherein the non-ionic surfactant is present at a concentration of 10-2000 μg/ml, such as 50-1000 μg/ml, 100-500 μg/ml or about 200 μg/ml.

30. An aqueous solution composition according to any one of claims 1 to 29, which additionally comprises a preservative such as a phenolic or benzylic preservative.

31. An aqueous solution composition according to claim 30, wherein the phenolic or benzylic preservative is selected from the group consisting of phenol, m-cresol, chlorocresol, benzyl alcohol, propyl paraben and methyl paraben.

32. An aqueous solution composition according to claim 30 or claim 31, wherein the preservative is present at a concentration of 10-100 mM, such as 20-80 mM or 25-50 mM.

33. An aqueous solution composition according to any one of claims 1 to 32, which is a composition for use in therapy.

34. An aqueous solution composition according to any one of claims 1 to 32, which is a pharmaceutical composition.
